(19) United States
(12) Patent Application Publication
(10) Pub. No.: US 2009/0192362 A1
(43) Pub. Date: Jul. 30, 2009

(54) System And Method For Corroborating Transitory Changes In Wellness Status Against A Patient Population

(76) Inventor: Robert J. Sweeney, Woodbury, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(21) Appl. No.: 12/352,469

(22) Filed: Jan. 12, 2009

Related U.S. Application Data
(60) Provisional application No. 61/023,324, filed on Jan. 24, 2008.

(51) Int. Cl. A61B 5/00

(52) U.S. Cl. 600/300

(57) ABSTRACT

A system and method for corroborating transitory changes in wellness status against a patient population through remote patient care is provided. A population that includes patients under remote patient care is managed. Physiometry for each patient over corresponding temporal periods is regularly obtained and contains recorded physiological measures. A wellness status for each patient is determined. The physiometry is evaluated against historical data maintained for the patient over a predetermined temporal period. Each difference in the physiometry and the historical data that exceeds a testing metric is flagged as a transitory change. Profiles of the patients in the population, which are similar, for those patients presenting corresponding transitory changes are correlated. A cumulative transitory change for the correlated patients is generated. The transitory change of each patient is corroborated against the cumulative transitory change to determine one of a match and deviation in wellness statuses.

### CROSS-REFERENCE TO RELATED APPLICATION

This non-provisional patent application claims priority under 35 U.S.C. §119(e) to U.S. Provisional Patent application, Ser. No. 61/023,324, filed Jan. 24, 2008, the disclosure of which is incorporated by reference.

### FIELD

The present invention relates in general to remote patient management and, specifically, to a system and method for corroborating transitory changes in wellness status against a patient population.

### BACKGROUND

Patient management is currently evolving towards a remote health care provisioning model under which patients suffering chronic disorders and other health conditions are managed out-of-clinic through a centralized caregiver data facility. Centralized remote health care is characterized by regular or on-demand uploads of local patient data, often on a daily basis, from a population of patients. Each patient uses a self-operable interrogator, commonly known as a “repeater” or “communicator.” Interrogators allow caregivers at a data facility to remotely gather physiological measures and other information from each patient, which can also be supplemented with interactive questioning regarding perceived health. In general, interrogators can directly retrieve data recorded by patient medical devices (PMDs) capable of remote interrogation, both implanted and external, which provide monitoring or therapy delivery, such as pacemakers, implantable cardioverter-defibrillators, and other devices.

In traditional clinic-based health care, patients are often seen on an infrequent basis and patient wellness is evaluated with particular emphasis on present physiologic indications as identified for that patient only. Remote health care, however, can provide a broader global view of a patient population that shares similar attributes and health characteristics, and data for the patient population can be queried and analyzed contemporaneously as an adjunct to an individual patient's evaluation. Remote health care thus affords a broader perspective and health-related phenomena and extrinsic influences and trends affecting multiple patients during the same period can be matched and identified.

Remote health care, though, removes a caregiver from one-on-one interpersonal patient interaction. A caregiver is thus deprived from making observations of extrinsic factors that may otherwise be noted during a traditional patient interview, such as physical demeanor or impairments. Conversely, by widening the scope of patients under care, remote health care allows caregivers to identify patterns of behavioral or physiologic change that are simultaneously affecting numerous patients in the population, for instance, an increase in respiratory complaints occasioned by environmental causes. While the causes of every change in patient population condition may not be certain from remotely monitored data alone, a population-observed change may nevertheless warrant consideration when remotely evaluating that same change in a particular patient. The global perspective therefore offers at least a partial substitute for the missing extrinsic factors.

The displaced nature of remote health care complicates determining whether a change in patient wellness is merely transitory, an indication of an acute medical condition, such as heart failure decompensation, or some other concern affecting patient well-being. The principle of hysteresis, as applied to patient care, counsels treating acute conditions more aggressively than a chronic worsening, yet the data available through remote care for a single patient may be insufficient to determine whether prompt action is needed. As a result, recognizing corresponding transitory changes in other patients with similar disorders can assist with resolving patient health status.

Existing remote health care, however, fails to weigh concurrent population-based changes into patient care. For instance, U.S. Patent Publication No. 2007/0198300, published Aug. 23, 2007, to Duckert et al., discloses computing a clinical trajectory of chronic disease patients. A database of de-identified patient data catalogs the patients by disease and attributes, and includes outcomes of adherence and non-compliance to treatment regimens. The database chronicles cumulative patient histories that are not applied to other patients. Rather, the de-identified patient data is used retrospectively to urge a patient with an on-going chronic disease to comply with a treatment regimen.

Similarly, U.S. Pat. No. 6,416,471, issued Jul. 9, 2002, to Kumar et al., discloses a portable remote patient telemonitoring device. An adhesive, cordless and disposable sensor band sends data to a signal transfer unit, either worn or positioned nearby a patient. A base station receives data transmissions from the signal transfer unit, which are sent to a remote monitoring station that provides presentation and review of the data. An operator call remotely program a base station to collect data from the patient, or can dial in to manually check on the status of a patient. However, any change in a patient's health condition is identified as a result of manual review of patient data on an individual patient basis.

Therefore, an approach is needed to provide patient evaluation against a database of dynamic population-based information, which would enable identification and consideration of physiometric changes simultaneously affecting multiple patients under remote care through use of a centralized data facility. Preferably, such an approach would buffer caregiver action in response to non-specific transitory changes to patient wellness.

### SUMMARY

A population of patients are remotely managed through a centralized data repository for health care. Physiometry, which can include qualitative data, if available, is regularly uploaded from one or more medical devices in use by each patient in the population. The physiometry is evaluated for transitory changes, which may have occurred during a recent temporal period, generally ranging over the near term. Each patient's wellness status is determined, which includes comparing any transitory changes to prior medical history. Any transitory changes that exceed a threshold or other testing metric, including non-thresholding criteria, are flagged as presenting matters of potential medical concern, such as an acute onset health condition. The patient's profile is correlated against the profiles of other patients in the population. The profile data for the correlated subpopulation is evaluated for similar transitory changes, which are compared to the patient's transitory change. Matches between a patient's transitory change and a similar transitory change in the correlated subpopulation indicate that the patient's change is statistically-likely to be due to extrinsic factors acting on both the patient and correlated subpopulation, and less likely to have been caused by strictly physiological reasons. For instance, a major snowstorm could force patients in a particular metropolitan region to stay home and thereby reflect a marked reduction in physical activity. A disparity between the respective transitory changes, though, may indicate a medical concern in the patient that requires some form of action or follow-up.

One embodiment provides a system and method for evaluating transitory changes in status during remote patient care. Physiometry is regularly obtained and contains recorded physiological measures for a population, which includes patients under remote patient care. The physiometry are evaluated to ascertain a wellness status of each patient. A transitory change in the wellness status of one of the patients is observed. A profile of the one patient to a set of other patients in the population, who each have a profile similar to the one patient, is correlated. The transitory change of the one patient is corroborated against a cumulative transitory change observed in the wellness statuses for the set of other patients.

A further embodiment provides a system and method for corroborating transitory changes in wellness status against a patient population through remote patient care. A population that includes patients under remote patient care is managed. Physiometry for each patient over corresponding temporal periods is regularly obtained and contains recorded physiological measures. A wellness status for each patient is determined. The physiometry is evaluated against historical data maintained for the patient over a predetermined temporal period. Each difference in the physiometry and the historical data that exceeds a testing metric is flagged as a transitory change. Profiles of the patients in the population, which are similar, for those patients presenting corresponding transitory changes are correlated. A cumulative transitory change for the correlated patients is generated. The transitory change of each patient is corroborated against the cumulative transitory change to determine one of a match and deviation in wellness statuses.

Still other embodiments will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a functional block diagram showing, by way of example, a remote patient health care environment.

FIG. 2 is a block diagram showing, by way of example, a schema for a database storing patient records.

FIG. 3 is a graph showing, by way of example, patient and population activity levels over corresponding time periods.

FIG. 4 is a process flow diagram showing a method for corroborating transitory changes in wellness status against a patient population, in accordance with one embodiment.

FIG. 5 is a data flow diagram showing the categories of patient data.

FIG. 6 is a process flow diagram showing patient status determination for use with the method of FIG. 4.

FIG. 7 is a data flow diagram showing factors affecting patient-to-population correlation.

FIG. 8 is a process flow diagram showing, by way of example, follow-up actions for use with the method of FIG. 4.

FIG. 9 is a block diagram showing for a system for corroborating transitory changes in wellness status against a patient population, in accordance with one embodiment.

### DETAILED DESCRIPTION

### Remote Patient Care Environment

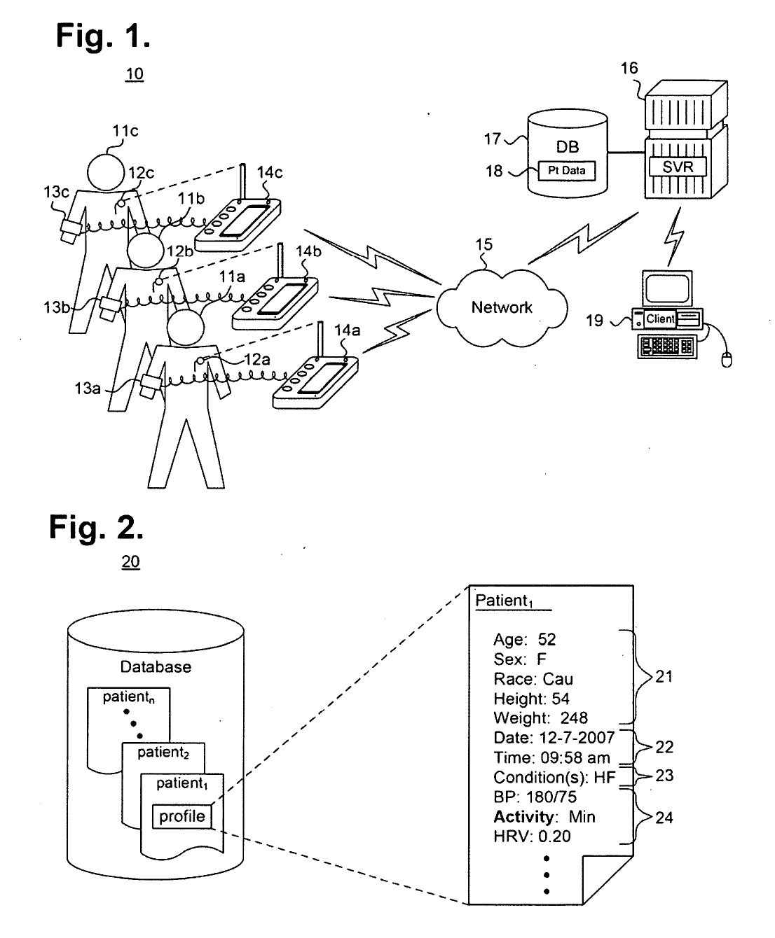

At a minimum, providing remote health care requires out-of-clinic patient information collection points and a centralized data facility, which serves as a data repository and wellness resource for caregiver use. FIG. 1 is a functional block diagram showing, by way of example, a remote patient health care environment 10. Individual patients 11a-c, who collectively constitute a patient population, receive remote health care from caregivers (not shown) through a centralized healthcare server 16. Each patient 11a-c uses a patient-operable communicator 14a-c, which can be remotely interfaced online to the server 16 over a public data communications network 15, such as the Internet. The network 15 operates under the Transmission Control Protocol/Internet Protocol (TCP/IP) protocol, although other network protocols could be used. In a further embodiment, the communicators 14a-c can be interfaced via a telephone line, including conventional land line and cellular connections, such as described in commonly-assigned U.S. Pat. No. 7,009,511, issued Mar. 7, 2006 to Mazar et al., the disclosure of which is incorporated by reference. Other network topologies and communication configurations are possible.

Each communicator 14a-c helps the server 16 to follow and monitor the physiometry and well being of each patient 11a-c, by uploading the physiometry to the server 16 on a regular basis or expressly requested, such as in response to a caregiver request. The patient physiometry is recorded by patient medical devices at a frequency applicable to device type or as required. Implantable medical devices, for instance, generally record physiological measures on continuous, periodic, event-driven, or binned average bases, while external medical devices may record physiological measures less frequently or on an event occurrence-basis. Other physiological data measure recordation time frames are possible. Additionally, qualitative data, such as self-assessments of activities of daily life and subjective responses of patient condition, may also be uploaded, if available. Henceforth, “physiometry” will refer to quantitative physiological measures, qualitative subjective data, and any other form of uploadable patient-related information, except as indicated otherwise.

The patient physiometry may be obtained through external sensors 13a-c, which can be integral to or connectable to the communicator 14a-c by wired or wireless means, such as inductive telemetry, radio frequency (RF) telemetry, or other wireless telemetry based on, for example, “strong” Bluetooth or IEEE 802.11 interfacing standards. Other types of external sensor connections are possible. In addition, the external sensors may continuously remain in contact with a patient's body, such as the sensor used with a Holter monitor, or be of a type that the patient can manually use, operate, or upon which he can perform extracorporeal testing, such as a blood pressure cuff, weight scale, blood glucose meter, Spiro meter, skin resistance sensor, and the like. Still other external sensors are possible.

Patient physiometry can also be obtained and recorded directly through the sensors operated by IMDs 12a-c that are permanently or temporarily introduced into a patient's body. Internal sensors can be provided integral or connected to implantable medical devices 12a-c (IMDs), such as the sensors found on device leads for providing sensing within and for delivering cardiac therapy to a patient's heart. IMDs may be fully implanted into a patient's body, which includes therapy delivery devices, such as pacemakers, implantable cardioverter-defibrillators, biventricular pacemakers, and neuro-stimulators; and physiometric monitoring devices, such as cardio or pulmonary monitors. IMDs also include fully implanted dedicated sensors, such as a pulmonary artery pressure sensor. IMDs may also be partially introduced into a patient's body, which includes physiometric monitoring devices, such as interstitial blood glucose monitors that pair a disposable subdural catheter connected to an external wearable control unit. Other types of IMDs are possible.

Recorded physiometry is collected as patient data 18 and centrally stored by the server 16 into a database 17, as further described below with reference to FIG. 2. The server 16 is a server-grade computing platform configured as a uni-, multi- or distributed processing system, which includes those components conventionally found in computing devices, such as, for example, a central processing unit (CPU), memory, network interface, persistent storage, and components for interconnection. Care givers, including physicians, nurses, and other medical staff, as well as researchers and authorized personnel, can access the patient data 18 and other information, including analyses and reports, through client devices 19, such as personal computers, or from other interconnected computing platforms (not shown).

In a further embodiment, the patient data 18 is extracorporeally safeguarded through security and cryptographic measures against unauthorized disclosure to third parties, including during recordation, collection, assembly, evaluation, transmission, and storage, to protect patient privacy and comply with medical information privacy laws, such as the Health Insurance Portability and Accountability Act (HIPAA) and the European Privacy Directive. At a minimum, patient health information that identifies a particular individual with health- and medical-related information is treated as protectable, although other kinds of sensitive information in addition to or in lieu of specific patient health information could also be protectable.

### Patient Population Data

The database 17 provides a persistent electronic data repository of both historical and on-going physiometry for the patient population. FIG. 2 is a block diagram showing, by way of example, a schema for a database 17 storing patient records. The schema can be implemented as a flat, relational, hierarchical, or other form of structured data store.

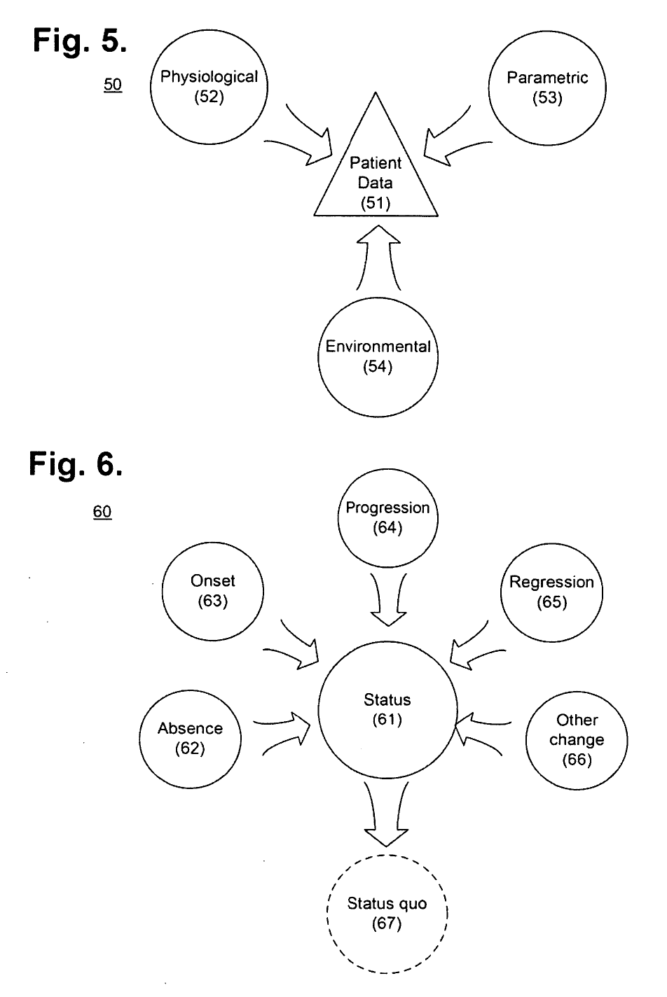

The patient data 18 is continually updated as new patient physiometry is regularly uploaded from communicators 14a-c. Patient data 18 can also be supplemented with data provided from other sources, such as data collected during an in-clinic visit, laboratory testing, or hospitalization. The broad categories of patient data 18 are further described below with reference to FIG. 5. The patient data 18 includes patient records containing a profile 20 of the attributes and health characteristics of each patient 11a-c. Attributes 21 include the age, gender, race, weight, height, and general patient information. Health characteristics include data on temporality 22, such as the date and time of corresponding physiometry 24, and on a health condition or disorder 23 applicable to the patient, if any. The health condition 23 can include both the primary disorder and any comorbidities. Other patient data 18 is possible.

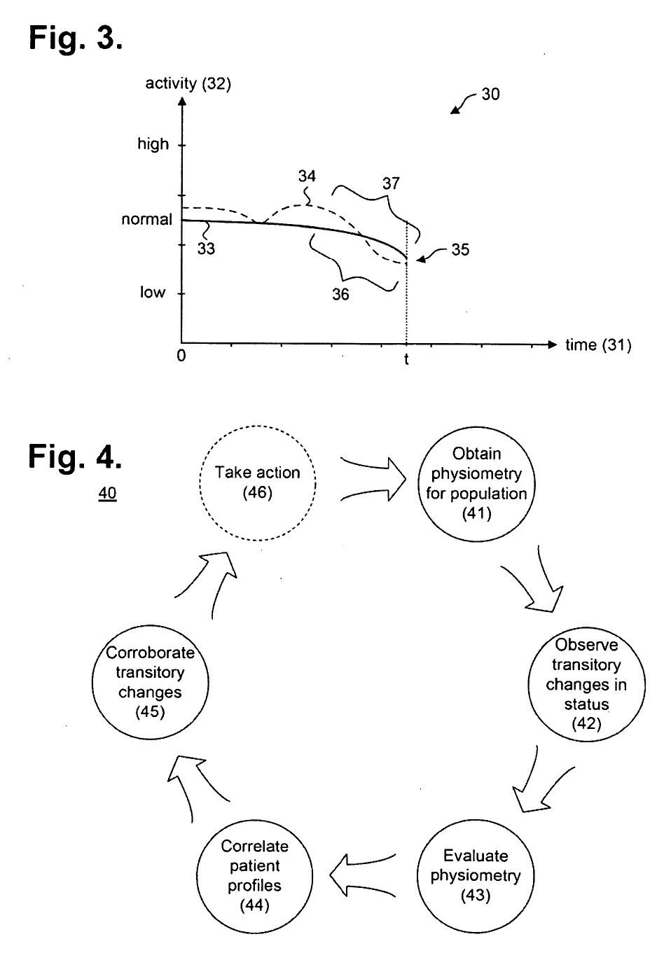

The temporality data 22 facilitates population-based comparisons, which is central to evaluating apparently transitory changes in individual patients. FIG. 3 is a graph 30 showing, by way of example, patient and population activity levels 34, 35 over corresponding time periods. The x-axis 31 represents time with a resolution D, and the y-axis 32 represents levels of patient activity.

In one embodiment, a time resolution D, of 24-hours can be used, and patient activity can be normalized over a qualitative range of High-Normal-Low. At each time resolution point, the levels of patient activity for each patient in the population are evaluated and normalized to the qualitative scale. At any particular time resolution point, a patient will reflect either a high, normal, or low activity level. Other time resolutions and patient activity levels are possible.

Other types of patient-to-population comparisons are also possible, including different time resolutions for each patient and comparing changes in population physiometry over a sliding time window, which can be backward- or forward-adjusted per patient or subpopulation. As well, other patient-to-population comparison metrics besides patient activity are possible, like physiological indications, coughing, sneezing or congestion.

A transitory change 36 reflected in numerous patients in the population sharing similar attributes and health characteristics as a subpopulation may help explain a transitory change presenting in a particular patient. For example, a marked decrease in a patient's activity level 33 may be innocuous, or be indicative of an acute condition, or other matter of possible medical concern or follow-up. A transitory change 37 echoed as a corresponding decrease in the activity level 34 of the population as a whole can clarify the nature of the change for that patient.

At a specific time t, a patient's profile 20 can be correlated to that of other patients in the population and both the patient's and the correlated population's activity levels can be compared. Any observed difference 35 between the two activity levels can be used to corroborate, or dispel, the existence of behavioral or physiologic change in that patient due to extrinsic factors. As a caution, the propriety of relying solely on a corroborating population-observed transitory change to clarify or discount an individual patient's transitory change depends upon the magnitude of the difference 35 and other factors, as further described below with reference to FIG. 4. For instance, in isolation, a decrease in patient's activity level could signal impending heart failure decompensation. However, a heavy snowfall that temporarily restricts the activities of all patients living in the same geographic region would explain why the population's activity level decreased during a time period concurrent with the patient's activity level decrease.

### Method

Transitory changes in patient wellness status can present at any time for patients in a population under remote health care. A transitory change 36 for a single patient could indicate the need for undertaking action or follow-up. The patient data 18 enables transitory changes 37 occurring to a matching part of the patient population during the same time period to be identified and compared. FIG. 4 is a process flow diagram showing a method 40 for corroborating transitory changes in wellness status against a patient population, in accordance with one embodiment. The method 40 is performed as a series of process steps by the server 16, or general purpose programmable computing device having access to the database 17.

Patient population monitoring and evaluation is performed as a continuous cycle (operations 41-45). During each iteration of the cycle, physiometry is obtained for each patient 11a-c in the population (operation 41). The physiometry is collected by the server 16 on a regular basis, or as requested or required. The physiometry can either be “pushed” to the server 16 by each communicator 14a-c (shown in FIG. 1) or “polled” by the server from each communicator. Physiometry collection could also be triggered by a patient event, including a sensed physiological or behavioral health condition, or by manual patient or caregiver initiation.

For each patient 11a-c, any transitory changes 36 in status are observed (step 42), which can include identifying an actionable change of some type in the health condition of the patient, including one of a status quo, progression, regression, onset, and absence of the health condition, such as described in commonly-assigned U.S. Patent Pub. No. US-2008-0021287, published on Jan. 24, 2008, the disclosure of which is incorporated by reference. A transitory change can be an observable increase, decrease, or other change in a physiological measure of sufficient magnitude to raise possible medical concern, which has occurred within a relatively short temporal period. The magnitude of change and observation time frame will depend upon both the type of physiological measure and whether other physiological measures also reflect changes during the same period.

In general, the physiometry for a particular patient i can be represented as a two-dimensional M×N matrix, where there are M patients in a patient population [symbol] and there are N measures known for a particular type of physiology n. A physiological measure for patient i recorded at time t can be expressed as a tuple, such that:

P(i,t)=P(i,T₀+jDₜ) (1)

where iε{M:m is a patient, such that S(t)ₘ}, T₀ is an initial time, j is the number of the times that the physiological measure has been recorded, Dₜ is a time resolution, and S(t)ₘ={s₀,s₁,s₂, . . . ,sₜ₋₁}, which is a stream of recorded measures for a physiology of type n, where nεN.

An aggregate physiological measure for a patient population [symbol] recorded at time t can be similarly expressed as a singleton, such that:

$$POP ( t ) = {\sum }_{i = 1}^{M} \frac{P ( i , {T}_{0} + {jD}_{t} )}{M} \qquad ( 2 )$$

where POP(t) is an average of individual physiological measures for all of the patients in the population.

For an individual patient, a transitory change observed at time t can be expressed as the difference between the current value and an average of the cumulative values of a physiological measure, such that:

$$Δ P ( i , t ) = P ( i , t ) - {\sum }_{k = {L}_{1}}^{{L}_{2}} \frac{P ( i , {T}_{0} + ( j - k ) {D}_{t} )}{{L}_{2} - {L}_{1} + 1} \qquad ( 3 )$$

where L₁ and L₂ represent a temporal period starting at L₁ and ending at L₂, such that L₁≦j and L₂≦j.

For a patient population, a transitory change observed at time t can be expressed as the difference between the current aggregate value and an average of the cumulative aggregate values of a physiological measure, such that:

$$Δ POP ( t ) = POP ( t ) - {\sum }_{k = {L}_{1}}^{{L}_{2}} \frac{POP ( {T}_{0} + ( j - k ) {D}_{t} )}{{L}_{2} - {L}_{1} + 1} \qquad ( 4 )$$

Other representative metrics in lieu of averaging, including mean, median, and standard deviation, could also be used.

In the broadest sense, any physiologic change presenting over a short time period constitutes a transitory changes. The transitory change need not be physically observable to the human eye and may present wholly through physiometry recorded through sensors integral to or connected with implantable or external patient medical devices. Thus, if ΔP(i,t) is non-zero, a transitory change 36 has occurred, which can be Generalized, such that:

|ΔP(i,t)|≧s (5)

where s is a threshold applicable to the physiological measure. Accordingly, any transitory change ΔP(i,t) exceeding the threshold s is considered medically significant and possibly actionable. Similarly, if ΔPOP(t) is non-zero, a transitory change 37 has occurred, which can also be generalized, such that:

|ΔPOP(t)|≧s (6)

Other determinations of transitory change, temporal period, and medical significance, including non-threshold-based tests, are possible. A threshold test is generally unidimensional and a measure either is within or in excess of the threshold at a specific point in time. Modest changes may not exceed the threshold when observed in isolation, yet may cumulatively exceed the threshold, albeit over an extended time period. As well, a plurality of individual physiological measures could be evaluated and a conclusion of a transitory change may be reached when a majority of the physiological measures exhibit some change in value, whether thresholded or simply different than when last recorded.

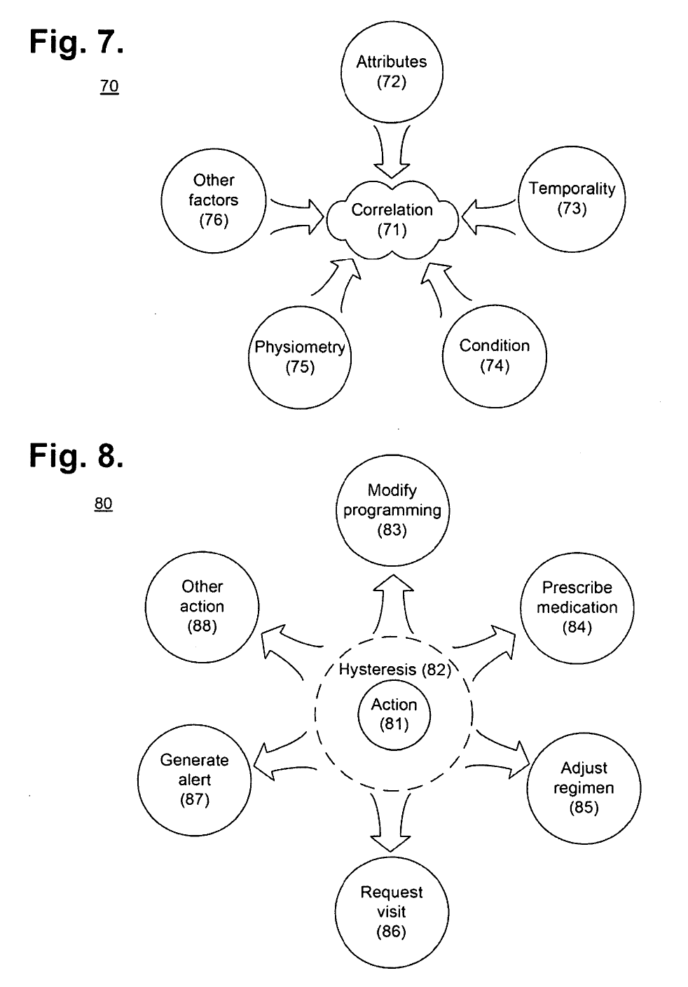

Upon observing a transitory change 36 in a patient, the set of uploaded physiometry and, if applicable, qualitative data is evaluated to determine a patient wellness status (operation 43), as further described below with reference to FIG. 6. If the transitory change 36 appears to be of possible medical concern, that patient's profile 20 is correlated to the profiles of other patients in the population (operation 44). Correlation involves identifying other patients in the population who share similar attributes and health characteristics, as further described below with reference to FIG. 7. In a further embodiment, the patient's profile 20 is only correlated and subsequently compared if the transitory change 36 indicates an abnormal condition, such as where one or more of the physiological measures is in an abnormal range for that value.

Finally, the transitory change 36 is corroborated against any transitory changes 37 observed in those patients identified as having correlated profiles 20 (operation 45). An individual patient's transitory change ΔP(i,t) can be directly compared to the correlated population's transitory change ΔPOP(t) to find any correspondence between the two changes, such that:

ΔABS(i,t)=ΔPOP(t)−ΔP(i,t) (7)

Consequently, ΔABS(i,t)→0, that is, a near-zero difference between the transitory changes, indicates that the patient's transitory change ΔP(i,t) closely matches the correlated population's transitory change ΔPOP(t) and could be explainable by extrinsic factors similarly affecting the patient population.

The extrinsic factors need not necessarily be identified as part of the inquiry and correlation to the patient subpopulation. Rather, a close matching of the patient's transitory change ΔP(i,t) to the correlated population's transitory change ΔPOP(t) can alone suffice. In some situations, extrinsic factors may be known, either based on patient data 18 (shown in FIG. 1) or by virtue of other knowledge provided to the server 16 from sources other than tile communicator 14a-c and their respective implantable or external patient medical devices. Conversely, where ΔABS(i,t) is large, other reasons for the patient's transitory change ΔP(i,t) may apply, such as an onset of an acute health condition, and, if appropriate, diagnosis and follow-up actions can be taken (operation 46), as further described below with reference to FIG. 8. Other patient population monitoring arid evaluation operations are possible.

Patient Data Categories

Patient data can originate from IMDs 12a-c, external sensors 13a-c, and other sources, such as the communicators 14a-c or other servers, and can be grouped into three categories. FIG. 5 is a data flow diagram showing the categories 50 of patient data 51. The categories are non-exclusive and not meant to be exhaustive, and include physiological measures category 52, parametric data 53, and environmental data 54 categories. The physiological measures category 52 include physiometry recorded by patient medical devices, and can also be supplemented with qualitative data. The parametric data category 53 reports the status and operational characteristics of a patient medical device, such as programming or battery status. Finally, the environmental data category 54 includes non-patient and non-device related information, such as the ambient temperature and time of day. Other patient data categories are possible.

Patient Status Determination

In general, a patient's wellness status will vary slightly from day-to-day. However, a sudden large change, gradual changes that have cumulatively grown past a certain point, and multiple concurrently occurring changes to different types of physiometry could each signify a medically significant event, which each must be considered in light of a transitory change. FIG. 6 is a process flow diagram showing patient status determination 60 for use with the method 40 of FIG. 4. The patient's wellness status 61 reflects an assessment of whether any transitory change in the primary disorder or status quo, that is, no change, applies. Other outcomes from the evaluation are possible.

Moreover, a patient's wellness status is a shorthand term for referencing the holistic overall physical and mental well-being of a patient. The wellness status thus can include an assembly of quantitative and qualitative measures, both directly recorded and subsequently derived, that collectively constitute the patient data 18 (shown in FIG. 1) from which medical diagnoses, including identifying an actionable change of some type in the patient's health condition, including one of the status quo, progression, regression, onset, and absence of the health condition.

Additionally, one or more changes of physiometry could present in a set of uploaded physiological measures and, if applicable, qualitative data. A single physiometric change is usually not sufficient to draw a medical conclusion. However, that same single physiometric change could be corroborated through diagnosis with other physiometric changes in the patient, which, taken together, signal a medical event occurrence. For example, angina, when accompanied by either a reduction in exercise capacity or respiratory distress, can indicate a potential onset of myocardial ischemia when conditioned on a time course of short duration. Accordingly, the physiometry associated with the transitory change, as well as other related patient data, can be evaluated for an absence 62, onset 63, progression 64, or regression 65 of one or more chronic or acute health conditions, such as described in commonly-owned U.S. Pat. No. 6,336,903, to Bardy, issued Jan. 8, 2002; U.S. Pat. No. 6,368,284, to Bardy, issued Apr. 9, 2002; U.S. Pat. No. 6,398,728, to Bardy, issued Jun. 4, 2002; U.S. Pat. No. 6,411,840, to Bardy, issued Jun. 25, 2002; and U.S. Pat. No. 6,440,066, to Bardy, issued Aug. 27, 2002, the disclosures of which are incorporated by reference. Other changes 66 in patient status 61 are possible, such as a change in a comorbidity. As well, maintenance of status quo 67 might be notable.

Patient-to-Population Correlation

Correlation ensures that a transitory change in a particular patient is compared to only those transitory changes in other patients in the population who are sufficiently similar and potentially influenced by the same, albeit possibly unknown, extrinsic factors. FIG. 7 is a data flow diagram showing factors 70 affecting patient-to-population correlation. The factors 70 follow from the patients' profiles to include attributes 72 and health characteristics of each patient 11a-c, including temporality 73, health condition or disorder 74, and physiometry 75. No one factor is fully dispositive in correlating the patient to a matching part of the population and depend upon the nature of the transitory change. Other factors 76 are possible. For example, a marked increase in respiratory distress may be due to poor air quality in a certain city and the attributes 72 that identify a patient's locale would be of greater weight However, an increase in heart rate might be untied to geographical boundaries, yet be presented in those patients having a particular health condition 74, such as heart failure. The simultaneity of the condition 74 and the temporality 73 of the heart rate increase might signal that an extrinsic factor is at play, such as a nationwide news event, like a national election. Thus, correlation requires matching the transitory change to other similar transitory changes occurring within the same general temporal period, followed by a further inquiry into other factors that may cause the patient to be similar, or dissimilar, to those patients matched from the population.

Follow-Up Actions

Corroborating an individual patient's transitory change against the transitory chances of a population serve to buffer immediate interventive action in response to the change. However, when follow-up is needed, following diagnosis, a range of actions are possible. FIG. 8 is a process flow diagram showing, by way of example, follow-up actions 80 for use with the method 40 of FIG. 4. One or more actions 81 may apply, and can be performed remotely or as instigated via the server 16, or other device. For instance, the programming of an IMD or external device might be modified remotely 83. A caregiver may decide to prescribe new medication or dosing instructions 84, adjust a treatment regimen 85, or request a patient visit in-clinic 86. Tile server 16, or other device, may unilaterally generate an alert 87 for caregiver or patient consideration. Still other actions 88 are possible.

In a further embodiment, hysteresis 82 is applied to the form of action 81 chosen in response to a transitory change of medical concert. Hysteresis is an important consideration when considering a transitory change that may, absent other evidence, resemble an acute medical condition. Hysteresis proportions the degree of action by weighing the temporality of the change. Thus, an acute change is treated more aggressively than a gradual change, which could require little or no additional action.

### System

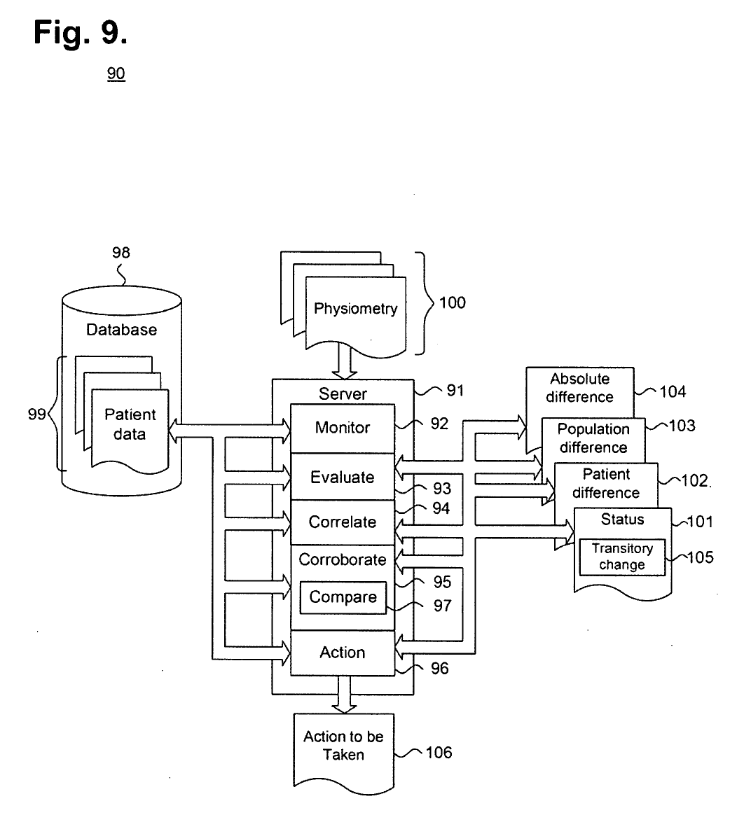

Transitory changes in a patient population are monitored and evaluated by a system having access to the database of regularly uploaded patient data. FIG. 9 is a block diagram showing for a system for corroborating transitory changes in wellness status against a patient population 90, in accordance with one embodiment. A centralized server 91 generally performs the monitoring and evaluation, but other systems or processing platforms are possible.

In one embodiment, the server 91 includes modules to monitor 92, evaluate 93, correlate 94, corroborate 95, and take action 96, as appropriate. The server 91 is coupled to a database 98 or other form of structured data store, within which patient data 99 is maintained. Other modules are possible.

The monitor module 92 regularly obtains physiometry 100 from IMDs 12a-c, external sensors 13a-c, and other sources, which is saved as patient data 99 into a corresponding patient record in the database 98. The physiometry 100 can be requested, or “pulled,” from each data source, or unilaterally sent or “pushed.” In addition, the physiometry 100 may be forwarded from an intermediary device, such as IMD-originated physiometry, which has been locally interrogated by a communicator that is uploading the physiometry to the server 91. Other physiometry monitoring arrangements are possible.

The evaluate module 93 observes any transitory changes 105 in each new set of physiometry 100 and generates a patient status 101, which represents an evaluation or, when sufficiently comprehensive, a diagnosis that can include the identification of an absence, onset, progression, or regression of one or more chronic or acute health conditions, as applicable. Each of the physiological measures and, if available, qualitative values, are compared to historical patient data 99 through the database 98 over a predetermined temporal period, which can include a sliding workflow of time. Each of the differences 102 in physiometry for that patient are tested against a threshold or other testing metric, including non-thresholding criteria, and those patient differences 102 that exceed the threshold or meet the criteria are flagged as a transitory change 105. The evaluate module 93 may also test for or diagnose chronic or acute health conditions. Other evaluation functions are possible.

The correlate module 94 attempts to match each transitory change 105 against transitory changes, if any, for other similarly situated patients in a subpopulation of the population at large. Those patients sharing similar attributes and health characteristics are identified as the applicable subpopulation and an aggregate difference 103 between the cumulative physiometry and historical patient data 99 for the correlated subpopulation is determined. The population difference 103 can be similarly tested against the threshold or other testing metric, including non-thresholding criteria, to identify whether the correlated subpopulation also presents a cumulative transitory change. Different patient subpopulations may apply for different transitory changes, even in the same patient. Other correlation functions are possible.

The corroborate module 95 determines the absolute difference 104 between the patient's transitory change 105 and the correlated subpopulation's transitory change, if any. In a further embodiment, a relative difference between the patient's transitory change 105 and a corresponding subpopulation's transitory change can be evaluated, as well as other comparative evaluations between a single patient's transitory change and a correlated subpopulation's transitory change. A compare submodule 97 compares the patient's and population's transitory chances, and matches and identifies deviations between the two transitory changes as reflected by absolute differences 104. A match indicates that the patient may be presenting a transitory change due to extrinsic factors, which are also affecting the correlated population. A match could also be explainable for reasons other than extrinsic factors, yet the statistically-supportable correlation between the same transient changes occurring in one patient and a subpopulation correlated by similar health characteristics and traits tend towards a conclusion that those individuals all have been affected by the same stimulus, which is likely extrinsic in nature. A divergence of transitory change, though, can indicate the possible onset of an acute condition or other medical concern. Other corroboration functions are possible.

Finally, the action module 96 chooses an action to be taken 106 if the patient's transitory change cannot be otherwise explained, or as required. The action need not be specifically performed by the server 91, and could instead be dispatched as instructions to a remote system or device, such as an communicator or IMD. The actions include modifying programming parameters, prescribing medication, adjusting a treatment regimen, requesting a patient visit, and generating an alert of the patient status 101. Other actions are possible. While the invention has been particularly shown and described as referenced to the embodiments thereof, those skilled in the art will understand that the foregoing and other changes in form and detail may be made therein without departing from the spirit and scope of the invention.

What is claimed is:

1. A system for evaluating transitory changes in status during remote patient care, comprising:
a database configured to store physiometry comprising recorded physiological measures, which has been regularly obtained for a population comprising patients under remote patient care; and
a server, comprising:
an evaluation module configured to evaluate the physiometry to ascertain a wellness status of each patient, and to observe a transitory change in the wellness status of one of the patients;
a correlation module configured to correlate a profile of the one patient to a set of other patients in tie population, who each have a profile similar to the one patient; and
a corroboration module configured to corroborate the transitory change of the one patient against a cumulative transitory change observed in the wellness statuses for the set of other patients.

2. A system according to claim 1, further comprising:
an analysis submodule configured to evaluate the recorded physiological measures against historical data of the one patient; and
a testing submodule configured to test each difference in the physiometry against a testing metric, and to flag each difference that exceeds the testing metric as the transitory change.

3. A system according to claim 1, wherein the wellness status is determined as one of an onset, progression, regression, and absence of a patient condition.

4. A system according to claim 1, further comprising:
an action module configured to undertake an action in respect of the one patient upon a divergence of the transitory change of the one patient and the cumulative transitory change for the set of other patients.

5. A system according to claim 4, wherein hysteresis is applied to the action in proportion to a degree of the transitory change.

6. A method for evaluating transitory changes in status during remote patient care, comprising:
regularly obtaining physiometry comprising recorded physiological measures for a population comprising patients under remote patient care;
evaluating the physiometry to ascertain a wellness status of each patient;
observing a transitory change in the wellness status of one of the patients;
correlating a profile of the one patient to a set of other patients in the population, who each have a profile similar to the one patient; and
corroborating the transitory change of the one patient against a cumulative transitory change observed in the wellness statuses for the set of other patients.

7. A method according to claim 6, further comprising:
evaluating the recorded physiological measures against historical data of the one patient;
testing each difference in the physiometry against a testing metric; and
flagging each difference that exceeds the testing metric as the transitory change.

8. A method according to claim 6, further comprising:
determining one of an onset, progression, regression, and absence of a patient condition as the wellness status.

9. A method according to claim 6, further comprising:
undertaking an action in respect of the one patient upon a divergence of the transitory change of the one patient and the cumulative transitory change for the set of other patients.

10. A method according to claim 9, further comprising:
applying hysteresis to the action in proportion to a degree of the transitory change.

11. A system for corroborating transitory changes in wellness status against a patient population through remote patient care, comprising:
a monitoring module configured to manage a population comprising patients under remote patient care, and to regularly obtain physiometry for each patient over corresponding temporal periods;
a database comprising the physiometry maintained as recorded physiological measures;
an evaluation module configured to determine a wellness status for each patient, comprising:
an analysis submodule configured to evaluate the physiometry against historical data maintained for the patient over a predetermined temporal period; and
a testing submodule configured to flag each difference in the physiometry and the historical data that exceeds a testing metric as a transitory IS change;
a correlation module configured to correlate profiles of the patients in the population, which are similar, for those patients presenting corresponding transitory changes, and to generate a cumulative transitory change for the correlated patients; and
a corroboration module configured to corroborate the transitory change of each patient against the cumulative transitory change to determine one of a match and deviation in wellness statuses.

12. A system according to claim 11, further comprising:
an action module configured to take action for each patient with the deviation in wellness status.

13. A system according to claim 12, wherein the action is selected from the group comprising modifying programming parameters, prescribing medication, adjusting a treatment regimen, requesting a patient visit, and generating an alert of the status of the one patient.

14. A system according to claim 11, wherein each transitory change is determined as one or more of an average, mean, median, and standard deviation.

15. A system according to claim 11, wherein a match in wellness status is attributed to extrinsic factors affecting the correlated patients.

16. A system according to claim 11, wherein the physiometry is selected from the group comprising physiological measures, parametric data, and environmental data.

17. A system according to claim 11, wherein the physiometry is provided from one or more of an implantable medical device and external medical device.

18. A method for corroborating transitory changes in wellness status against a patient population through remote patient care, comprising:
managing a population comprising patients under remote patient care;
regularly obtaining physiometry for each patient over corresponding temporal periods and comprising recorded physiological measures;
determining a wellness status for each patient, comprising:
evaluating the physiometry against historical data maintained for the patient over a predetermined temporal period; and
flagging each difference in the physiometry and the historical data that exceeds a testing metric as a transitory change;
correlating profiles of the patients in the population, which are similar, for those patients presenting corresponding transitory changes;
generating a cumulative transitory change for the correlated patients; and
corroborating the transitory change of each patient against the cumulative transitory change to determine one of a match and deviation in wellness statuses.

19. A method according to claim 18, further comprising:
taking action for each patient with the deviation in wellness status.

20. A method according to claim 19, wherein the action is selected from the group comprising modifying programming parameters, prescribing medication, adjusting a treatment regimen, requesting a patient visit, and generating an alert of the status of the one patient.

21. A method according to claim 18, further comprising:
determining each transitory change as one or more of an average, mean, median, and standard deviation.

22. A method according to claim 18, further comprising:
attributing a match in wellness status to extrinsic factors affecting the correlated patients.

23. A method according to claim 18, wherein the physiometry is selected from the group comprising physiological measures, parametric data, and environmental data.

24. A method according to claim 18, wherein the physiometry is provided from one or more of an implantable medical device and external medical device.
